(19) United States
(12) Patent Application Publication
(10) Pub. No.: US 2017/0241965 A1
(43) Pub. Date: Aug. 24, 2017

(54) KIT FOR DETECTING THE PRESENCE OF A FLUID

(71) Applicant: LogiCO2 International AB, Vastra Frolunda (SE)

(72) Inventor: Bo HANSSON, Mersch (LU)

(73) Assignee: LogiCO2 International AB, Vastra Frolunda (SE)

(21) Appl. No.: 15/051,388

(22) Filed: Feb. 23, 2016

(51) Int. Cl. G01N 33/00; G08B 5/22; B65D 81/113

(52) U.S. Cl. CPC G01N 33/0036; B65D 81/113; G08B 5/22

(57) ABSTRACT

The present invention relates to a kit for detecting the presence of a fluid, said kit comprising a portable device for sensing a fluid and communicating the presence of said fluid to a user, and a fluid permeable case comprising at least one internal cavity, wherein said at least one internal cavity is arranged to accommodate and releasably hold said portable device.

### FIELD OF THE INVENTION

The present invention relates to a kit for detecting the presence of a fluid, said kit comprising a portable device for sensing and communicating the presence of said fluid to said user, and a fluid permeable case comprising at least one internal cavity.

### BACKGROUND OF THE INVENTION

Devices for sensing and communicating the presence of fluids, for instance measuring the carbon dioxide concentration in a room or a space, are often mounted on the wall of a room. This creates a problem when a user would like to measure the concentration of a fluid in a space that contains no such device. For this reason, various types of portable devices for sensing and communicating the presence of fluids are available. However, while simply moving the device from one room to another might somewhat mitigate the problem in some cases, the fact remains that it could be dangerous to enter the room in which the presence of a fluid is to be sensed if the fluid in question is harmful. Such situations often require the user to bring specialized equipment for remotely sensing and communicating the presence of said fluid in said space, e.g. remotely controlled robots equipped with sensors, which can be very expensive and require expert skills to operate. For certain users, e.g. fire fighters and/or police officers, being able to sense the presence of a fluid in a space which is potentially dangerous to enter without having to bring expensive and specialized equipment is desirable.

### SUMMARY OF INVENTION

An object of the present invention is therefore to provide a kit which allows a user to detect the presence a fluid in a room which is potentially dangerous to enter.

A further object of the invention is to provide a kit for sensing and communicating the presence of fluids in a space which is adjacent to that which is occupied by the user, where said kit is both easy to use and cheaper to manufacture than the abovementioned presently available alternatives.

The above and other objects which will be evident from the following description are achieved by a kit for allowing a user to detect a fluid according to the appended claims.

According to one exemplary embodiment, a kit is provided for detecting the presence of a fluid, said kit comprising a portable device for sensing a fluid and communicating the presence of said fluid to a user, and a fluid permeable case comprising at least one internal cavity. Said at least one internal cavity is arranged to accommodate and releasably hold said portable device.

By providing the fluid permeable case with at least one internal cavity arranged to accommodate and releasably hold the portable device, the portable device can be protected from impacts while not interfering with its ability to detect the presence of a fluid. This has several benefits, e.g. a fireman can throw the kit down a flight of stairs before descending. Doing so lowers the risk of the fireman being exposed to dangerous levels of, for example, CO₂, as the presence of dangerous levels of CO₂ is sensed and communicated to the user before he/she descends the stairs.

The portable device being able to be releasably held by the at least one internal cavity means that the user is allowed to choose, for each situation, whether or not he/she would like to use the kit in its entirety or just use the portable device as it is. Another benefit of said portable device being arranged to be releasably held by said fluid permeable case is that the portable device is easier to carry if a user can remove the fluid permeable case when it is not needed. Having to carry a shockproof case even when it is not needed might unnecessarily impede the user's movements.

According to one exemplary embodiment, the portable device comprises at least one sensing unit being arranged to sense the presence of a fluid, a communication unit being configured to communicate the presence of a fluid to an external display unit, and a housing accommodating said at least one sensing unit and said communication unit.

The design of the sensing unit can be varied in order to allow the kit to detect the presence of different kinds of fluids and to detect different concentrations of fluids. For example, in one embodiment, the kit might be arranged to detect and communicate to a user the presence of CO₂ and/or CO, while in another embodiment the kit might be arranged to detect and communicate to a user the presence of O₂, N₂, CH₄ or any other gas which might be harmful to human beings in high enough concentrations.

In a similar fashion, the design of the communication unit can be varied so that it allows communication of the readings of the sensing unit to an external display via any combination of Bluetooth, WiFi, radio, free-space optical communication and/or other similar types of wireless communication. Also, having the communication unit communicate the readings of the sensing unit to an external display via wired communication is also conceivable. According to one exemplary embodiment, said external display unit can be a smartphone. According to another exemplary embodiment, said external display unit can be another external device, for example but not limited to a computer monitor, a TV-screen or a handheld computer. One purpose of said communication is that the user of the kit can receive sensory readings in real-time from the adjacent space which is being sensed, thus removing the need to first sense the presence of a fluid in a adjacent space, then retrieve the kit, after which the user can be made aware of the potential presence of a fluid.

According to one exemplary embodiment, the at least one sensing unit is arranged to measure the concentration of at least one gas in ambient air, said gas being a gas chosen from a group comprising carbon dioxide (CO₂), carbon monoxide (CO), oxygen (O₂), nitrogen (N₂) and methane (CH₄).

According to one exemplary embodiment, the communication unit is arranged for wireless communication with said external display unit. By arranging the communication unit to allow wireless communication with said external display unit, the risk of a user becoming entangled in for example communication wires is reduced.

According to one exemplary embodiment, the fluid permeable case further comprises a retrieval unit. This allows the user to repeatedly use the kit for sensing the presence of a fluid in a space adjacent to that which is occupied by the user, without ever having to enter said adjacent space. This is beneficial in the event that the kit, when the user throws or otherwise projects the kit towards said adjacent space, misses the intended destination and the kit instead lands somewhere inaccessible. In that case, the user is able to retrieve the kit by means of the retrieval unit and retry placing the kit in the adjacent space by throwing or otherwise projecting it. This is also beneficial in the event that the user wishes to sense the presence of a fluid in several different adjacent rooms before choosing which to enter. This is also beneficial in the event that the user wishes to descend a flight of stairs leading into a space which is potentially filled with harmful fluid. In that case, the user might use the retrieval unit in order to be able to sense the presence of a fluid on each landing before descending.

According to one exemplary embodiment, the retrieval unit comprises a rope, wire or tether arranged to connect said fluid permeable case to a reference point. Having the retrieval unit comprise a rope, wire or tether helps fulfill the purpose of the kit being cheap and easy to manufacture.

According to one exemplary embodiment, the retrieval unit is arranged as a wire connecting the portable device with the external display unit. Thus, there is no need for the retrieval unit and the communication unit to be arranged as two separate units. This decreases the design complexity of the kit and makes it cheaper and easier to manufacture.

According to one exemplary embodiment, the fluid permeable case is made of a soft material. By making the fluid permeable case of a soft material, the portable device is protected from impact damages when the kit is thrown or otherwise projected in a space adjacent to that which is occupied by the user. Soft material is herein defined as a material which, when compared with the material of the housing of the portable device, exhibits significant shock absorption properties and thus has a cushioning effect on said portable device.

According to one exemplary embodiment, the fluid permeable case further comprises a first portion and a second portion, said first portion and said second portion being removably attachable to each other. Having the fluid permeable case comprise a first portion and a second portion removably attachable to each other makes it easier to accommodate the portable device inside the fluid permeable case, thus making the assembly of the kit easier.

According to one exemplary embodiment, the fluid permeable case is elastically deformable so that it is storable inside a container which has an internal volume which is between 5%-60%, more preferably between 10%-50% and most preferable between 10%-35% of the volume of said fluid permeable case in its expanded state. The volume of the fluid permeable case is herein defined as the external volume of the fluid permeable case. If the fluid permeable case is elastically deformable as described above, the user will benefit from having a fluid permeable case which is easy to store when it is not being used.

According to one exemplary embodiment, the fluid permeable case is spherical. By having a fluid permeable case which has a spherical shape, it is possible for a user to roll the kit into the adjacent space in which the presence of a fluid is to be sensed, instead of throwing or otherwise projecting it towards said space. This further decreases the risk of the portable device suffering impact damages.

According to one exemplary embodiment, the fluid permeable case is made of a material chosen from the group of rubber, latex, nylon, polyurethane, polychloroprene, polyvinyl chloride (PVC) and/or any polymeric foam. Having the fluid permeable case made from any of these materials will help ensure that the fluid permeable case is sufficiently soft to protect the portable device from impact damages. These materials are also sufficiently flexible to allow a user to press fit the portable device in the at least one internal cavity of the fluid permeable case.

According to one exemplary embodiment, the fluid permeable case is arranged as an inflatable case provided with apertures connecting said cavity to ambient air. This is an alternative way to achieve the desired impact protection properties of the fluid permeable case, while at the same time allowing the portable device to sense the presence of a fluid. As impact damages are most likely to occur on the edges and corners of the portable device, leaving the faces of the portable device open to ambient air does not significantly decrease the impact protection the inflatable case offers. Also, having the fluid permeable case arranged as an inflatable case greatly increases its storability when it is not being used, as the deflated inflatable case only takes up a fraction of the volume of the inflated inflatable case.

According to one exemplary embodiment, the inflatable case is made of a material chosen from the group of rubber, latex, nylon, polychloroprene, and/or polyvinyl chloride. This enables the inflatable case to be flexible and allows the inflatable case to be repeatedly inflated and deflated without experiencing material failure.

According to one exemplary embodiment, the kit further comprises a container having an internal volume which is between 5%-60%, more preferably between 10%-50% and most preferable between 10%-35% of the volume of the fluid permeable case in its expanded state. Hence, the kit may also comprise a container in which the fluid permeable case may be stored in a compressed state.

According to one exemplary embodiment, said kit further comprises a container having an internal volume which is between 5%-60%, more preferably between 10%-50% and most preferable between 10%-35% of the volume of said fluid permeable case in its expanded state plus the volume of the portable device for sensing a fluid. According to this exemplary embodiment, the portable device for sensing a fluid and the fluid permeable case in a compressed state may be stored in the same container.

### BRIEF DESCRIPTION OF THE DRAWINGS

The above, as well as additional objects, features and advantages of the present invention, will be better understood through the following illustrative and non-limiting detailed description of exemplary embodiments of the present invention, with reference to the appended drawings, wherein:

FIG. 1 is an exploded view of a portable device being part of the present invention,

FIG. 2 is a perspective view of an unassembled kit according to the present invention, said kit comprising said portable device and a fluid permeable case,

FIG. 3a is another perspective view of said kit, in which said kit is assembled,

FIG. 3b is a cross-section of the first and second locking portions of the fluid permeable case in FIG. 3a,

FIG. 4a shows a container in which the fluid permeable case is stored when it is not used, and the fluid permeable case,

FIG. 4b shows a container in which the fluid permeable case and the portable device is stored when they are not used, and the fluid permeable case and the portable device,

FIG. 5 is a schematic view showing wireless communication between said portable device and a smartphone, and

FIG. 6 is a perspective view of a second embodiment of the fluid permeable case, being part of the present invention, and

FIG. 7 is a perspective view of a third embodiment of the fluid permeable case, being part of the present invention.

### DETAILED DESCRIPTION OF PREFERRED EMBODIMENTS

The invention will now by way of example be described in more detail by means of embodiments and with reference to the accompanying drawings.

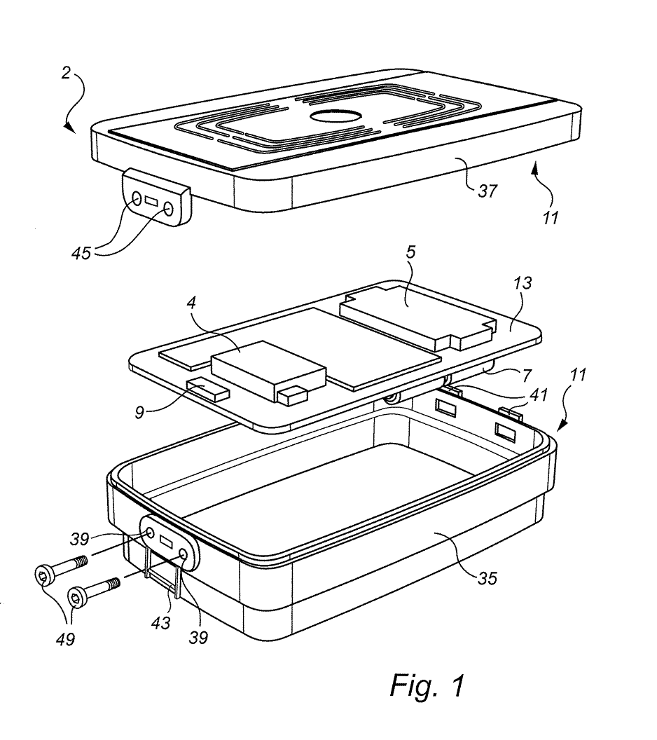

FIG. 1 is an exploded view of a portable device 2, said portable device 2 comprising a sensing unit 4 for sensing the presence of a fluid, a communication unit 5 for communicating the readings of the sensing unit 4 to an external display, a battery 7 for powering the portable device 2, and a housing 11. The portable device 2 further comprises a USB-port 9 for allowing a user to connect the portable device 2 to a computer in order to allow configuration of the portable device 2. The sensing unit 4, the communication unit 5, the battery 7 and the USB-port 9 are arranged on a circuit board 13 which is arranged inside the housing 11.

The housing 11 of the portable device 2 is a substantially rectangular box. In one exemplary embodiment, the housing 11 has a length of 90 mm, a width of 60 mm and a height of 20 mm. Said housing 11 comprises a first portion 35 and a second portion 37. The first portion 35 comprises screw holes 39, snap joints 41 and an anchor point 43, said anchor point 43 being arranged to allow a user to attach the portable device 2 to a band (not shown) which can be hung around the neck of the user when the portable device 2 is to be carried on the user's person. The first portion 35 of the housing 11 is arranged to accommodate the circuit board 13. The second portion 37 comprises screw holes 45 and is arranged to be attachable to the first portion 35 by means of screws 49 and snap joints 41, thereby fixing the circuit board 13 in place.

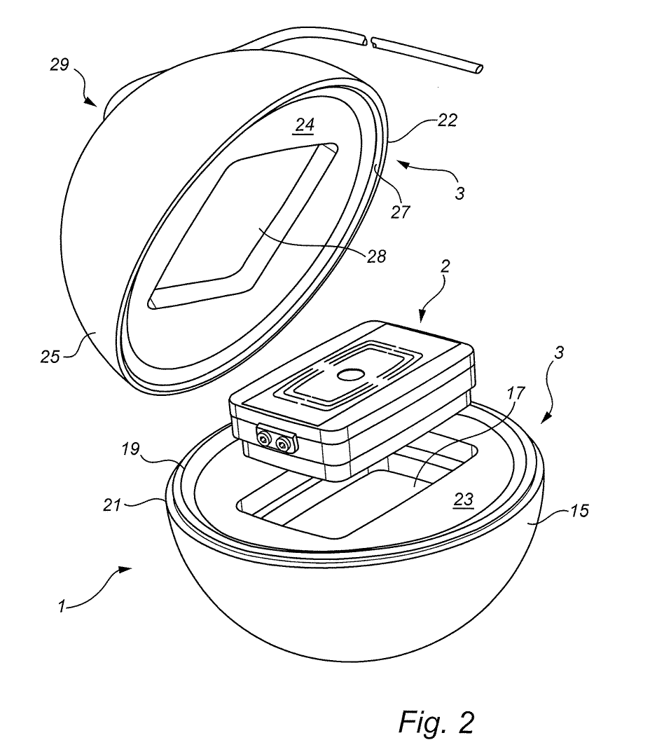

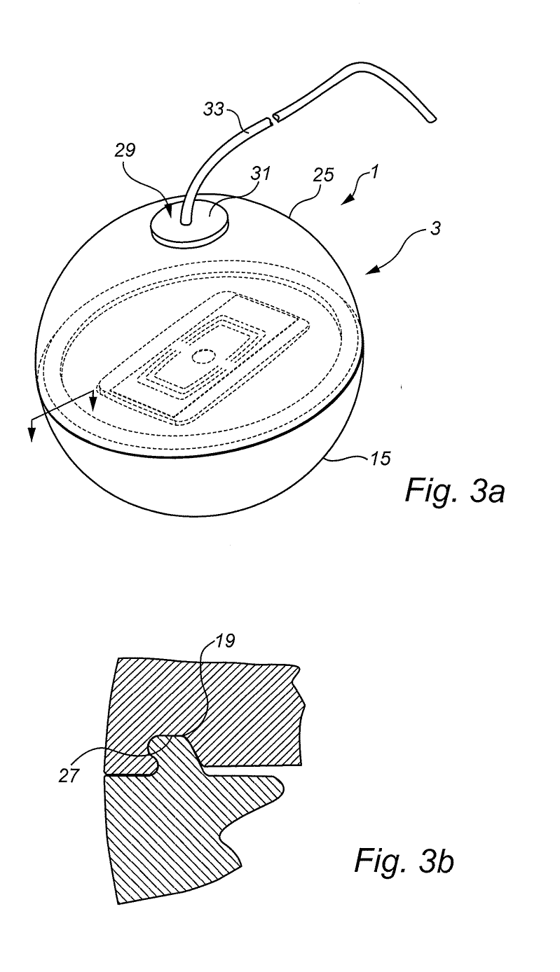

FIG. 2 is a perspective view of a kit 1 according to the present invention, said kit 1 comprising said portable device 2 and a fluid permeable case 3. In FIG. 2, the kit 1 is in an unassembled state, while in FIG. 3a the kit 1 is in an assembled state.

The fluid permeable case 3 shown in FIGS. 2 and 3a is made of polyurethane and is arranged as a split sphere. The fluid permeable case 3 thus comprises a first part 15 and a second part 25, both being substantially shaped as spherical caps. The first part 15 and the second part 25 comprises a first flat surface 23 and a second flat surface 24, respectively, arranged so that the two flat surfaces face each other when the first part 15 and the second part 25 are put together to form a sphere. The first part 15 further comprises an internal cavity 17 arranged to accommodate a part of and hold the portable device 2. The first part 15 also comprises a first locking portion 19 arranged on a rim 21 of the first flat surface 23.

The second part 25 comprises a second locking portion 27 arranged on a rim 22 of the second flat surface 23. The second part further comprises an internal cavity 28 arranged to accommodate a part of and hold the portable device 2. The second part 25 is arranged to be removably attached to the first part 15 by means of engaging said first locking portion 19 and said second locking portion 27, thus allowing the portable device 2 to be held in place between the first part 15 and the second part 25 of the fluid permeable case 3. The engagement of the first locking portion 19 and the second locking portion 27 is shown in greater detail in FIG. 3b, which shows a cross-section of the fluid permeable case 3 in FIG. 3a. In FIG. 3b, it is show that the first locking portion 19 is arranged as an annular protrusion. The second locking portion 27 is arranged as an annular recess arranged to accommodate the first locking portion 19 through press fit.

When inserting the portable device 2 into the fluid permeable case 3, as has been done in FIG. 3a, the portable device 2 is press fitted in internal cavity 17 and internal cavity 28. During this press fit, the fluid permeable case 3 is elastically deformed. The portable device 2 is then held in place by the friction exerted by the elastically deformed fluid permeable case 3 on the portable device 2.

In the assembled view in FIG. 3a, it is shown that when the first part 15 and the second part 25 of the fluid permeable case 3 are attached to each other, the portable device 2 is completely enveloped by the fluid permeable case 3. This causes the fluid performable case 3 to protect the portable device 2 from impact damage, e.g. damage caused by the kit 1 colliding with a hard surface after the user throws it or otherwise projects it into an adjacent space in order to sense the presence of a fluid there.

The fluid permeable case 3 is further arranged to be elastically compressible, meaning that it is made of a elastically deformable material having properties so that the fluid permeable case 3 can be compressed by applying pressure to it. As such, the fluid permeable case 3 can be seen as having an expanded and a collapsed state.

In FIGS. 2 and 3a, it is shown that the second part 25 of the fluid permeable case 3 further comprises a retrieval unit 29, said retrieval unit 29 comprising a mounting plate 31 attached to the second part 25 of the fluid permeable case 3 and a tether 33 attached to said mounting plate 31. The retrieval unit 29 is arranged to allow a user to retrieve the entire kit 1, e.g. from a room which is dangerous to enter, by pulling the tether 33.

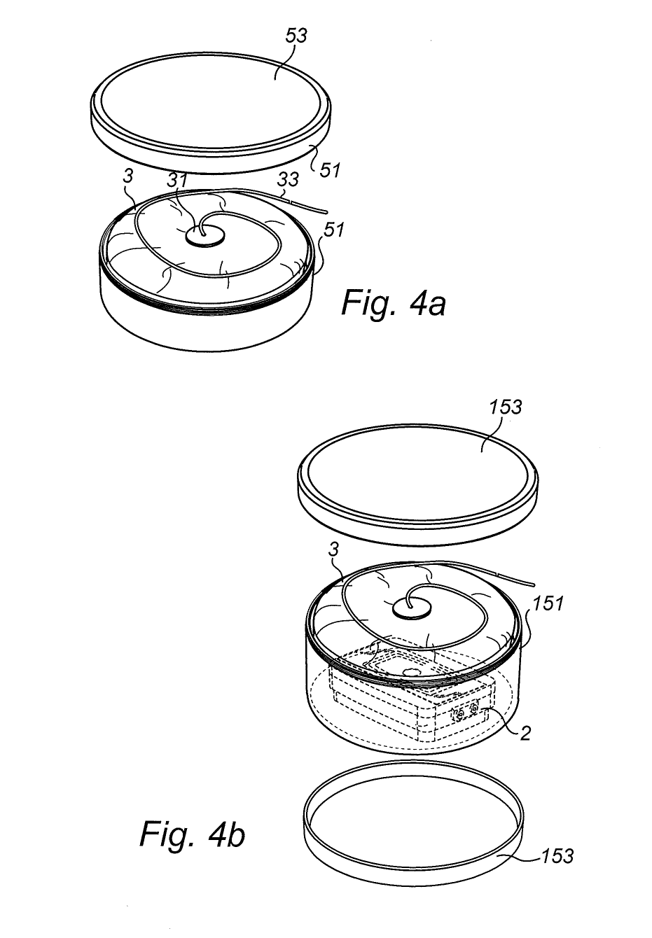

FIG. 4a is an exploded view showing a first embodiment of a container in which the fluid permeable case 3 may be stored when it is not used. The container 51 is arranged as a cylindrical box with a cap 53 covering the opening at the top. The cap is here illustrated as a screw cap, but other means for fastening the cap, such as a snap fit or a press fit, may also be used. The internal volume of the container 51 is significantly smaller than the volume of the fluid permeable case 3 in its expanded state. For example, the internal volume of the container 51 could be between 10% and 50% of the volume of the fluid permeable case 3.

FIG. 4b is an exploded view showing a second container in which the fluid permeable case 3 may be stored when it is not used. The difference between the first and the second embodiment is that in this second embodiment, also the portable device 2 may be stored in the same container as the fluid permeable case. The container 151 is arranged as a cylindrical cover with a cap 153 covering the opening at the top and a cap 153 covering the opening at the bottom. The caps are here illustrated as crew caps, but other means for fastening the caps, such as a snap fit or a press fit, may also be used. The internal volume of the container 151 is large enough to store the portable device 2 and the fluid permeable case 3 in a compressed state. For example, the internal volume of the container 151 could be between 10% and 50% of the volume of the fluid permeable case 3 plus the volume of the portable device 2.

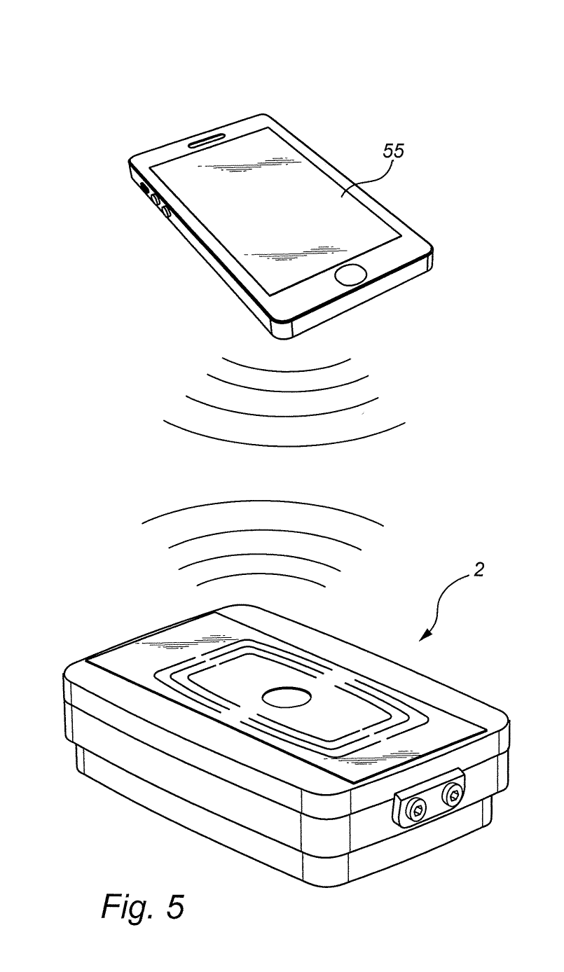

FIG. 5 is a schematic view showing wireless communication between the portable device 2 and a smartphone 55. Said wireless communication could for example be through the use of Bluetooth, WiFi, radio or free-space optical communication. The purpose of communicating the readings of the sensing unit 4 to an external display, e.g. said smartphone 55, is that the use of an external display removes the need for a display on the portable unit 2, thus making it lighter, cheaper and more durable,

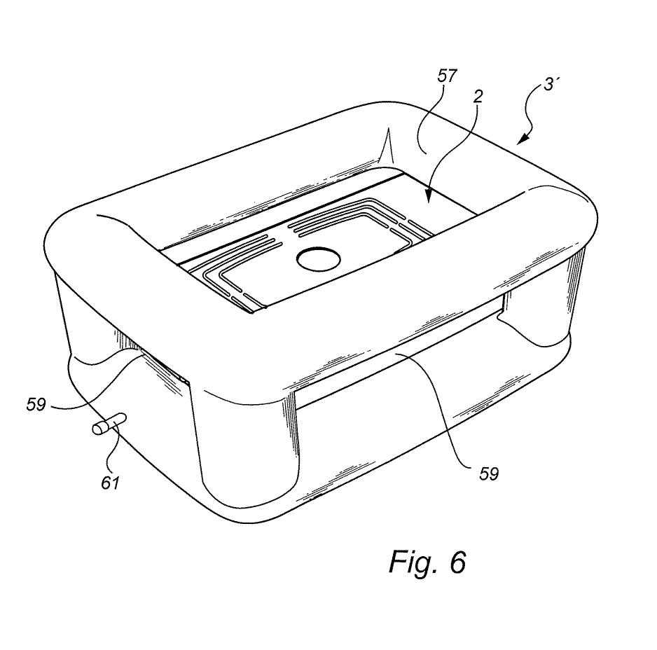

FIG. 6 illustrates another embodiment of the fluid permeable case. In FIG. 6, the fluid permeable case 3′ is arranged as an inflatable case 57 enveloping and protecting the portable device 2. In this embodiment, a valve 61 is provided, allowing a user to inflate and deflate the inflatable case 57. Before the inflatable case 57 is inflated, the portable device 2 can be inserted therein so that it is at least partly enveloped by said inflatable case 57. If, after the insertion of the portable device 2 into the inflatable case 57, the inflatable case 57 is inflated, then the portable device 2 is held in place by friction from the now inflated inflatable case 57 and is thus protected from impact damages. When inflated, the inflatable case 57 protects the edges and corners of the portable device 2 and is provided with apertures 59 for allowing a fluid to come into contact with the portable device 2.

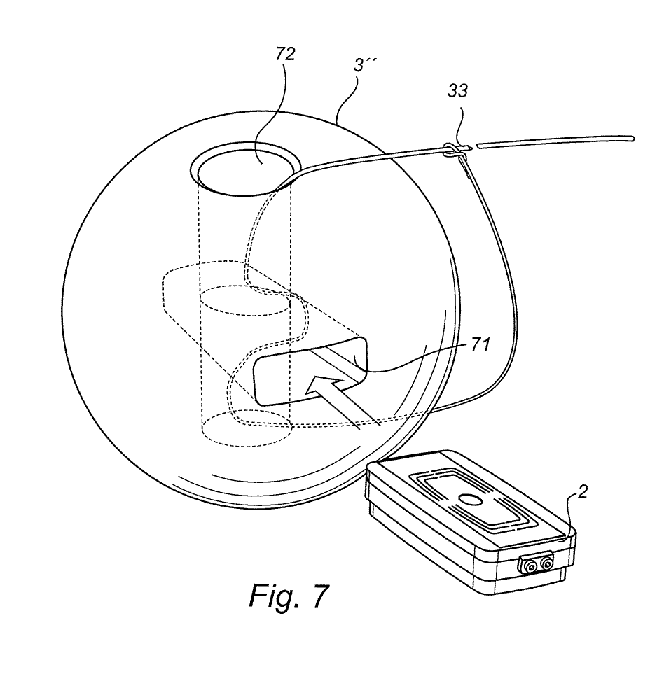

FIG. 7 illustrates a third embodiment of the fluid permeable case. In FIG. 7, the fluid permeable case 3″ is made of polyurethane and in one piece. It has a first opening 71 and a second opening 72. Both the first and the second openings 71, 72 may be produced by punching.

The first opening 71 is provided at a great circle or orthodrome of the spherical fluid permeable case 3″. Hence, the opening 71 extends from the surface of the fluid permeable case 3″ and into and through its center. The opening 71 has a general rectangular shape, which corresponds to the shape of the portable device 2. There is also a general correspondence between the cross-section area of the portable device 2 and the opening 71. The opening 71 is illustrated as a trough-hole, but it may also end inside the fluid permeable case 3″. The relevant size is that it protrudes sufficiently long into the fluid permeable case to fit the entire portable device 2 therein so that the portable device becomes protected by the fluid permeable case. When an operator intends to use the kit, he may then push the portable device into the opening 71, where it is held by press-fit and use the kit as described for the previous embodiments.

The second opening 72 is intended for connection of the retrieval unit 29. In this embodiment, the second opening 72 is also illustrated as a through hole, and a tether 33, being part of the retrieval unit 29, may then be inserted through the opening 72 and tied to itself. Also the second opening is provided at a great circle or orthodrome of the spherical fluid permeable case 3″. Hence, the opening 71 extends from the surface of the fluid permeable case 3″ and through its center. It is however provided at another great circle, such that it is a distance between the mouth of the first opening and the mouth of the second opening.

Although exemplary embodiments of the present invention have been described herein, it should be apparent to those having ordinary skill in the art that a number of changes, modifications or alterations to the invention as described herein may be made. Thus, the above description of the various embodiments of the present invention and the accompanying drawings are to be regarded as non-limiting examples of the invention and the scope of protection is defined by the appended claims.

For example, the containers 51 and 151 have been illustrated together with the fluid permeable case 3. However, the same containers may be used together with the inflatable fluid permeable case 3′ and the permeable case 3″.

Also, the fluid permeable cases 3 and 3″ have been illustrated as spherical. It is however not necessary and many other shapes are conceivable as long as they fulfill the purpose of holding and protecting the portable device 2.

Furthermore, any reference signs in the claims should not be construed as limiting the scope.

1. A kit for detecting the presence of a fluid, said kit comprising:
a portable device for sensing a fluid and communicating the presence of said fluid to a user, and a fluid permeable case comprising at least one internal cavity, wherein said at least one internal cavity is arranged to accommodate and releasably hold said portable device.

2. The kit according to claim 1, wherein said portable device comprises:
at least one sensing unit being arranged to sense the presence of a fluid,
a communication unit being configured to communicate the presence of a fluid to an external display unit, and
a housing accommodating said at least one sensing unit and said communication unit.

3. The kit according to claim 2, wherein said at least one sensing unit is arranged to measure the concentration of at least one gas in ambient air, said gas being a gas chosen from a group comprising carbon dioxide, carbon monoxide, oxygen, nitrogen, and methane.

4. The kit according to claim 2, wherein said communication unit is arranged for wireless communication with said external display unit.

5. The kit according to claim 1, wherein said fluid permeable case further comprises a retrieval unit.

6. The kit according to claim 5, wherein said retrieval unit comprises a rope, wire or tether arranged to connect said fluid permeable case to a reference point.

7. The kit according to claim 1, wherein said fluid permeable case is made of a soft material.

8. The kit according to claim 1, wherein said fluid permeable case further comprises a first portion and a second portion, said first portion and said second portion being removably attachable to each other.

9. The kit according to claim 1, wherein said fluid permeable case is elastically deformable so that it is storable inside a container which has an internal volume which is between 5%-60% of the volume of said fluid permeable case in its expanded state.

10. The kit according to claim 1, wherein said fluid permeable case is spherical.

11. The kit according to claim 1, wherein said fluid permeable case is made of a material chosen from the group of polyurethane, rubber, latex, nylon, polychloroprene, polyvinyl chloride and/or any polymeric foam.

12. The kit according to claim 1, wherein said fluid permeable case is arranged as an inflatable case provided with apertures connecting said cavity to ambient air.

13. The kit according to claim 12, wherein said inflatable case is made of a material chosen from the group of rubber, latex, nylon, polychloroprene, and/or polyvinyl chloride.

14. The kit according to claim 1, wherein said kit further comprises a container having an internal volume which is between 5%-60% of the volume of said fluid permeable case in its expanded state.

15. The kit according to claim 1, wherein said kit further comprises a container having an internal volume which is between 5%-60% of the volume of said fluid permeable case in its expanded state plus the volume of the portable device for sensing a fluid.
